(19) United States
(12) Patent Application Publication
(10) Pub. No.: US 2010/0174365 A1
(43) Pub. Date: Jul. 8, 2010

(54) MITRAL ANNULOPLASTY RING

(76) Inventors: Roberto Erminio Parravicini, Modena (IT); Alessandro Verona, Milano (IT)

Correspondence Address: DAVIDSON BERQUIST JACKSON & GOWDEY LLP, 4300 WILSON BLVD., 7TH FLOOR, ARLINGTON, VA, 22203, US

(21) Appl. No.: 12/449,601

(22) PCT Filed: Feb. 15, 2007
(86) PCT No.: PCT/IT2007/000102

§ 371 Date: Feb. 26, 2010

(51) Int. Cl. A61F 2/24

(52) U.S. Cl. 623/2.36

(57) ABSTRACT

A mitral annuloplasty ring composed of a D-shaped annular structure and made of rigid or semi-rigid material, a coating of the annular structure made of inert tissue and suited to be fixed with sutures to the heart tissues, and a pair of movable and sliding metal or plastic strips positioned in a folded conformation inside the annular structure and having a first end fixed to a rectilinear or circular portion of the annular structure and a second end resting on a curved portion of the annular structure.

### TECHNICAL FIELD

The present invention concerns a mitral annuloplasty ring.

### BACKGROUND ART

The mitral valve is the valve at the entrance of the left ventricle. The oxygenated blood arriving from the lungs collects in the left atrium and from there, through the mitral valve, it passes into the left ventricle which will then pump it into the whole body. The valve's function is to prevent the blood returning into the left atrium during ventricular contraction.

The mitral valve is composed of two valve flaps connected with “ties”, called tendinous cords, which, like the cords of a parachute, connect the flaps to the ventricular muscle (papillary muscles). The function of the tendinous cords is to regulate the apposition of the two flaps of the valve so that they meet perfectly during closing, preventing the backflow of blood.

The prolapse of the mitral valve is one of the commonest causes of malfunction of the valve. The prolapse of one or both of the valve flaps, due to the stretching or breakage of the tendinous cords, may, depending on the degree and extent, determine an imperfect closure and therefore cause a backflow of blood into the left atrium. According to the known technique, the pathology described above is tackled surgically by means of a sequence of operations which contemplates cutting a segment of the prolapsing rear flap, the subsequent reconstruction of the flap and the resuturing to the annulus of the valve, and finally suturing a reinforcing prosthetic ring around the annulus of the valve to stabilise it.

Although the surgical solution has proven effective, the need is nevertheless felt for a simpler and even more effective solution.

### DISCLOSURE OF INVENTION

The present invention concerns a mitral annuloplasty ring comprising an annular structure made of rigid or semi-rigid material and a coating of said annular structure made of inert tissue and suited to be fixed with sutures to the heart tissues; said ring being characterised by comprising stable deforming means positioned inside said annular structure; said stable deforming means comprise at least one folding strip positioned in a folded conformation inside the annular structure and having at least one end fixed to said annular structure; said strip being suited to be deformed and to maintain the imposed deformation, and said annular structure being suited structurally to undergo the deformations of said strip.

In this way, the surgeon who has to position the ring on the mitral valve may adapt its dimensions to the specifical needs of the patient by adjusting the fold of the strip.

### BRIEF DESCRIPTION OF THE DRAWINGS

The following example is given as illustration without limitation, for a better understanding of the invention with the aid of the figures in the enclosed drawing, in which:

FIGS. 1-5 illustrate in layout the realisation and the use of the ring of the present invention; and

FIG. 6 illustrates the ring in FIGS. 1-5 implanted in its working position corresponding to the mitral valve.

### BEST MODE FOR CARRYING OUT THE INVENTION

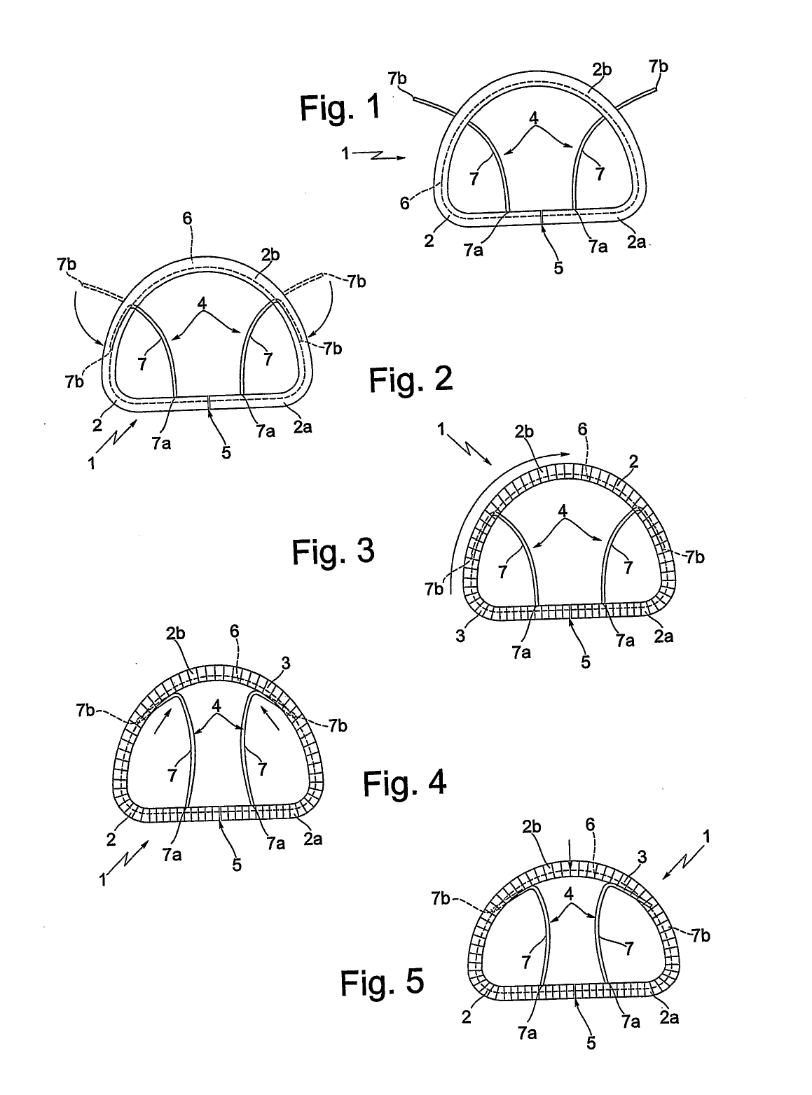

FIGS. 1-5 indicate as a whole with 1 a mitral annuloplasty ring according a to a preferred embodiment.

The ring 1 comprises an annular structure 2 made of plastic, a coating 3 of the annular structure 2 and two stable deforming elements 4. The annular structure 2 is made of plastic material and is substantially D-shaped, defined by a rectilinear portion 2a and a curved portion 2b. The annular structure 2 presents an opening 5 made in the linear portion 2a and a groove 6, illustrated with a dashed line in FIGS. 1-5, the function of which will be described below.

The coating 3 is made of inert tissue and has the aim of allowing the ring 1 to be fixed with sutures to the heart tissues.

Each of the two stable deforming elements 4 consists of a strip 7, which is made of metal or plastic material able to be deformed and to maintain the imposed deformation. Each of the strips 7 presents a first end 7a fixed to the rectilinear portion 2a, and a second end 7b resting on the inside of the groove 6.

The implementation of the ring 1 can be understood from the sequence in the FIGS. 1-5. In particular, once the two strips 7 have been fixed to the portion 2a of the annular structure 2 (FIG. 1), they are folded (FIG. 2) until the respective free ends 7b fit into the groove 6 where they rest (FIG. 3). As illustrated in FIG. 3, the annular structure 2 is covered with a coating 3 which, as stated above, is fixed with sutures to the heart tissues.

FIGS. 4 and 5 illustrate how it is possible to modify the extension of the annular structure 2 by adjusting the conformation of the strips 7. In particular, FIG. 4 and FIG. 5 show how it is possible respectively to lift and lower the curved portion 2b of the annular structure 2.

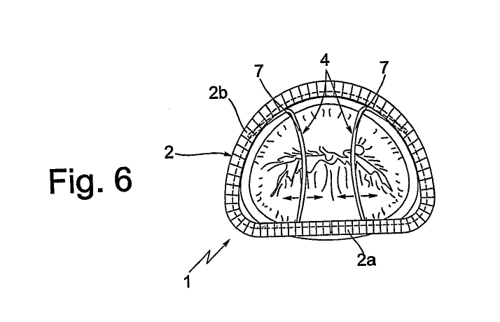

FIG. 6 illustrates the ring 1 implanted in left atrium corresponding to the mitral valve to correct its position with respect to the flaps. In particular, the ring 1 is positioned in such a way that the curved portion 2b of the annular structure 2 is located at the rear of the mitral valve to reduce the relaxation of the respective flap and to restore the correct functionality of the mitral valve itself.

The ring 1 of the present invention allows a simple solution of the problem concerning the prolapse of the mitral valve. Specifically, thanks to the combination of a deformable metal or plastic element that maintains the given deformation and of an annular structure inside which is placed said deformable element made of material such as to undergo structurally the deformations of the deformable strip itself, the ring 1 offers the advantage that it can be modified as desired depending on the specific implanting necessities.

Unlike the above description, the annular structure may present a completely closed circular shape in which the first ends 7a of the strips 7 are fixed to a curved section of the annular structure 2. Moreover, the annular structure 2 may be made of any other rigid or semi-rigid material and, even when D-shaped, it may have a closed structure and therefore without the opening in the rectilinear portion 2a.

1. A mitral annuloplasty ring comprising an annular structure made of rigid or semi-rigid material and a coating on said annular structure made of inert tissue and suited to be fixed with sutures to heart tissues; said ring comprising a deforming mechanism positioned inside said annular structure comprising at least one strip positioned in a folded conformation inside said annular structure and having at least one end fixed to said annular structure; said at least one strip being suited to be deformed and to maintain the imposed deformation, and said annular structure being suited structurally to undergo the deformations of said at least one strip.

2. The mitral annuloplasty ring according to claim 1, characterised in that said annular structure presents a groove in which rests a second free end of said strip.

3. The mitral annuloplasty ring according to claim 1, wherein the at least strip comprises two strips.

4. The mitral annuloplasty ring according to claim 3, wherein the strips are made of metal or plastic material.

5. The mitral annuloplasty ring according to claim 4, wherein the annular structure presents a D-shaped conformation having a rectilinear portion on which are fixed the first ends of the two strips, and a curved portion on which rest the second ends of the two strips.
